(19) United States
(12) Patent Application Publication
(10) Pub. No.: US 2021/0144966 A1
(43) Pub. Date: May 20, 2021

(54) SYSTEM AND METHOD FOR ACQUIRING INDIVIDUAL INFORMATION

(71) Applicant: ULIKEKOREA CO., INC., Seoul (KR)

(72) Inventor: Hee Jin KIM, Seoul (KR)

(21) Appl. No.: 16/950,830

(22) Filed: Nov. 17, 2020

(30) Foreign Application Priority Data
Nov. 19, 2019 (KR) 10-2019-0148593
Sep. 28, 2020 (KR) PCT/KR2020/013193

(51) Int. Cl. A01K 11/00; G08C 17/02

(52) U.S. Cl. CPC A01K 11/008; A01K 11/007; A01K 11/004; G08C 17/02

(57) ABSTRACT

The present disclosure relates to a system and method of acquiring individual information, and more particularly, to a system and method of effectively acquiring bioactivity and location information from each individual of livestock grazing in vast grasslands and efficiently utilizing the acquired information for livestock management by constructing a sensor-communication network.

The present application claims priority based on Korean Patent Application No. 10-2019-0148593, filed on Nov. 19, 2019 and PCT Application No. PCT/KR2020/013193, filed on Sep. 28, 2020, both of which are incorporated herein by reference in their entireties.

### BACKGROUND

### Field of the Disclosure

Embodiments relates to a system and method for acquiring individual information, and more particularly, to a system and method for acquiring individual information including location and bioactivity information data of livestock.

### Discussion of the Related Art

In general, when raising livestock such as cows and pigs, there may be serious damage due to the occurrence of various diseases related to breeding livestock such as foot-and-mouth disease or mad cow disease. One of the causes of these diseases is a narrow barn space. Such environment may weaken the immunity of livestock.

In this respect, as the importance of grazing livestock increases, cases of grazing and rearing livestock on vast grasslands rather than narrow barns are increasing.

However, in the case of grazing livestock in vast grasslands, there is a problem in that it is difficult to grasp and manage the location or state of livestock, and various methods have been attempted to specify the location of livestock. As representative methods, a GPS sensor is attached to livestock and then their location is tracked, or an aircraft such as a drone is launched and then its location is identified through image information.

Prior Patent 1 (KR10-1652192B), Prior Patent 2 (KR10-1536059B), and Prior Patent 3 (AU199736502B2) disclose obtaining location information of an individual by attaching an in vitro identification device to a livestock. Prior Patent 4 (EP0646313A1) and prior patent 5 (US2009/0182207A1) employ a method of inserting a sensor into body, but these prior patents have failed to acquire GPS data from an identification device smoothly, and disclosed a technology for effectively collecting data from livestock in a large area.

### SUMMARY

In addition to the individuals of the present disclosure as mentioned above, additional individuals and features of the present disclosure will be clearly understood by those skilled in the art from the following description of the present disclosure.

According to an embodiment of the present invention, a system for acquiring individual information including bioactivity and location information from two or more individuals comprises: a first identification device inserted in the body of each individual to acquire bioactivity information of the individual; a second identification device attached to the outside of each individual's body to acquire location information of the individual and to receive the bioactivity information acquired by the first identification device for generating individual information including the bioactivity and location information; and a relay network to receive individual information including the bioactivity and location information from the second identification device.

The second identification device further receive bioactivity and/or location information from other second identification devices of other individuals.

The relay network includes the second identification device of each individual.

The first identification device has an activation mode and an inactivation mode, and switches between the activation mode and the inactivation mode according to a control signal transmitted from the second identification device.

The inactivation mode of the first identification device has a power cut-off mode in which power is completely cut-off, and the power cut-off mode is switched to the activation mode due to a power transmission signal transmitted from the second identification device.

The second identification device transmits its own individual information along with individual information received from other second identification devices to the relay network.

The first identification device has an activation mode and an inactivation mode, and switches between the activation mode and the inactivation mode according to a predetermined time period.

According to the embodiments of the present disclosure, the second identification device further receives bioactivity information from one or more other first identification devices of other individuals.

According to an embodiment of the present invention, a method for acquiring individual information including bioactivity and location information from two or more individuals comprises: acquiring bioactivity information of each individual by a first identification device inserted in the body of the individual (bioactivity information-acquiring step); acquiring location information of the individual and receiving the bioactivity information from the first identification device by a second identification device attached to the outside of each individual's body for generating individual information including the bioactivity and location information (individual information-generating step), and transmitting the generated individual information to a relay network (individual information-transmitting step).

The individual information-transmitting step further comprises receiving individual information from other second identification devices of other individuals and transmitting it.

The method further comprises activating the first identification device prior to the bioactivity information-acquiring step (first identification device-activating step).

The first identification device-activating step receives an activation command including a power transmission signal, and the first identification device converts the power transmission signal into power and supplies power required to activate the first identification device.

According to the embodiments of the present disclosure, the individual information-transmitting step further comprises receiving individual information from one or more other second identification devices of other individuals and transmitting it.

In addition to the effects of the present disclosure as mentioned above, additional effects and features of the present disclosure will be clearly understood by those skilled in the art from the following description of the present disclosure.

### DETAILED DESCRIPTION

### Technical Problem

Accordingly, an individual of the present invention is to provide a sensor-communication network which is capable of stably acquiring location and bioactivity information data including physiological variables data of livestock, and efficiently utilizing the acquired location/bioactivity information data.

### Effect of the Disclosure

According to some embodiments, location and bioactivity information can be effectively acquired from each individual of livestock grazing in vast grasslands, and the information thus acquired can be effectively utilized for livestock management even in areas where commercial communication networks are not established or are not smoothly operated.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a schematic diagram showing the overall configuration of a system for acquiring individual information;

FIG. 2 is a schematic block diagram showing a detailed configuration of a first identification device;

FIG. 3 is a schematic block diagram showing a detailed configuration of a second identification device;

FIG. 4 is a schematic diagram showing a basic structure of data, which is identification information data, that is transmitted from a first identification device to a second identification device;

FIG. 5 is a schematic diagram showing a basic structure of data transmitted from a second identification device to a relay network;

FIG. 6 is a flowchart illustrating a method for acquiring individual information;

FIG. 7 is a view showing an embodiment in which first and second identification devices are attached to livestock (cows);

FIG. 8 illustrates an exemplary part of a configuration of a system for acquiring individual information;

FIG. 9 illustrates an exemplary overall configuration of a system for acquiring individual information;

FIG. 10 is a schematic block diagram showing a detailed configuration of a second identification device;

FIG. 11 illustrates an exemplary specific example of identification data transmitted from a first identification device to a second identification device; and

FIG. 12 illustrates an exemplary specific example of identification data transmitted from a second identification device.

### DETAILED DESCRIPTION OF THE DISCLOSURE

Hereinafter, embodiments of the present invention will be described in detail with reference to the accompanying drawings so that one with ordinary skill in the art may easily implement the present invention. However, the present invention may be implemented by various variations and is not limited to the embodiments described herein. In the drawings, parts irrelevant to the description are omitted such that the present invention may be clearly described, and similar reference numerals are used to similar parts throughout the specification.

Terms including ordinal numbers, such as first and second, may be used to describe various elements, but these elements are not limited by such terms. The terms are used only for the purpose of distinguishing one element from another element. For example, without departing from the scope of the present invention, a first element may be referred to as a second element, and similarly, a second element may be referred to as a first element. The terms used in this specification are used only to describe specific embodiments and are not intended to limit the present invention. Singular expressions include plural expressions unless the context clearly indicates otherwise.

Throughout this specification, a part “connected” to another part includes not only a part “directly connected”, but also a part “electrically connected” through another element. In addition, when a part “comprises” a certain element, it means that the part may also comprise other elements unless specifically stated to the contrary. As used herein, the term “a step . . . ” or “a step of . . . ” is not intended to mean “a step for . . . ”.

The terms used in this specification are general terms that are currently widely used in the art, but this may vary with the intention or precedent by one with ordinary skill in the art, the emergence of new technologies, and the like. In addition, in certain cases, terms are arbitrarily selected and used by the applicant, where the meaning will be described in detail in the description of the invention. Therefore, the terms used in this specification should be defined based on the meaning of the term and the overall contents of the present invention, not a simple name of the term.

The terminology of bioactivity information may include various biometrics, biometric data, bio-data, etc.

Hereinafter, the present invention will be described in detail with reference to the accompanying drawings.

### 1. System for Acquiring Individual Information According to the Invention

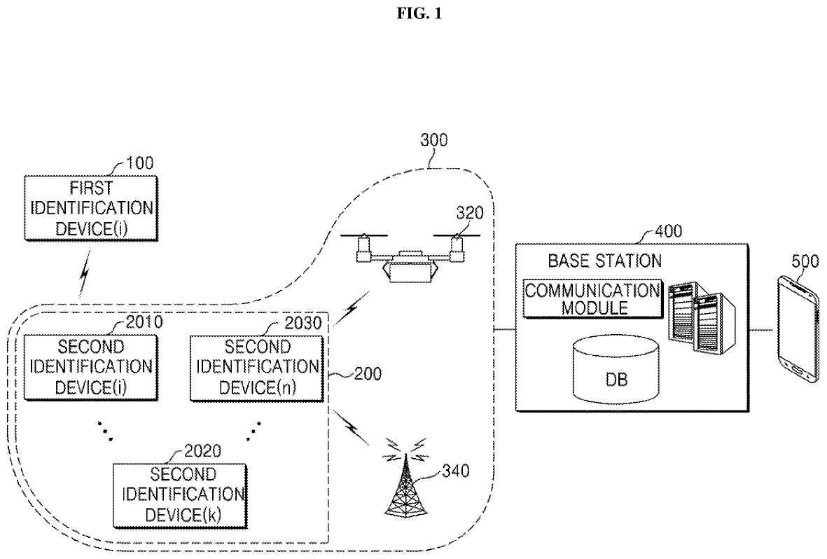

FIG. 1 is a schematic diagram showing the overall configuration of a system for acquiring individual information according to some embodiments. Referring to FIG. 1, the system for acquiring individual information comprises a first identification device 100 for acquiring bioactivity information of each individual, a second identification device 200, 2010, 2020, or 2030 for acquiring location information, the amount of activity, or internal and external temperature attached to each individual (livestock). a relay network 300 for receiving the bioactivity/location information, etc. of the individual from the first and second identification devices and uploading it on a communication network, a base station system 400 for receiving and integrating data from the relay network, one or more clients (for example, a first and second clients) 500 that access said system, a data processing server, and the like.

### 1.1. First Identification Device 100

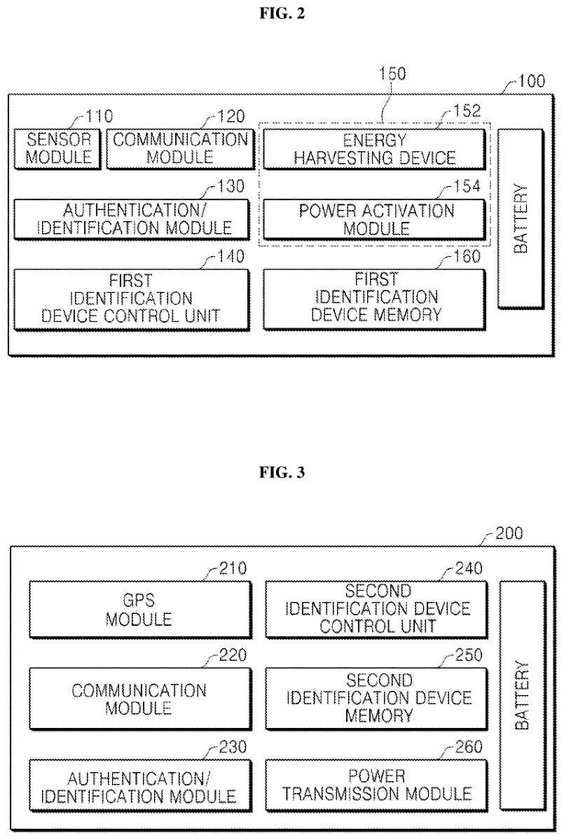

According to some embodiments, a first identification device is inserted in the body of each individual to measure bioactivity information of the individual and acquire the corresponding data. As shown in FIG. 2, the first identification device comprises a sensor module 110, a communication module 120, an authentication/identification module 130, a first identification device control unit 140, and a power control module 150 including an energy harvesting module 152, and a first identification device memory 160.

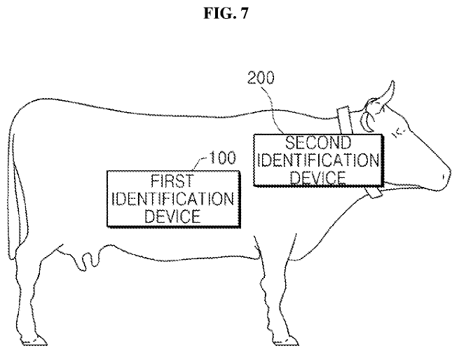

As shown in FIG. 7, the first identification device is inserted in the body of each individual, and may be a capsule form containing a sensor module 110. Examples of the first identification device may include biosensor capsules having shapes and structures as disclosed in Korean Patent Application No. 10-2016-0149744 by the applicant.

The first identification device may measure bioactivity information by settling specifically in the ruminant stomach such as first or second stomach in the body of each individual. According to some embodiments, the individual may be a ruminant animal that chews the cud, for example, a cow. In general, the stomach of a cow consists of the first (rumen), the second (reticulum), the third (Omasum), and the fourth (Abomasum). The first and the second are also called the “Reticulo-rumen” or simply the “Rumen.”

Among them, the first stomach is composed of several spaces, and accounts for 80 percent of the total volume of a stomach of a ruminant animal. The first stomach consists of a structure that includes the second stomach. The first or second stomach is the stomach where most of the important biological activities of the cow, such as digestive functions, are carried out. Therefore, the first identification device can be settled in the first or second stomach of the individual and obtains bioactivity information. The first identification device may be installed to settle in the second stomach. Due to the self-weight of the first identification device, the form of the second stomach, and the biological structure of the second and first stomachs, the first identification device may not be ejected well outside the second stomach after settling in the second stomach.

The system according to some embodiments comprises a plurality of first identification devices 100 (first identification device (i), i=1, 2, 3 . . . , k=the total number of livestock).

A bio-sensor capsule, an example of a first identification device, may contain one or more sensors. The first identification device may include an acceleration sensor to detect the acceleration value of the stomach of the cow, a gyro sensor to detect the angular velocity value of the stomach of the cow, a temperature sensor to detect the temperature value of the stomach of the cow, a methane sensor to detect the amount of methane gas produced in the stomach of the cow, and/or a PH sensor to detect the PH concentration of the stomach of the cow.

### A. Sensor Module 110

A sensor module may be configured by a plurality of sensors for acquiring bioactivity information in the body of the individual. The sensor module (110) may include a temperature sensor to measure the temperature of an individual's body temperature or ruminant.

The sensor module (110) may include an acceleration sensor, etc. that can measure the movement of an individual or the amount of activity of a rumen.

The first identification device (e.g., sensor module 110) may include an acceleration sensor to detect the acceleration value of the stomach or the activity of the cow, a gyro sensor to detect the angular velocity value of the stomach of the cow, a temperature sensor to detect the temperature value of the body of the cow, a methane sensor to detect the amount of methane gas produced in the stomach of the cow, and/or a PH sensor to detect the PH concentration of the stomach of the cow.

### B. Communication Module (First Individual Communication Module, 120)

It is a communication module to transmit data acquired from the sensor module to the outside.

A technology using a general public wireless communication network has conventionally been disclosed, but there is a disadvantage in that data transmission is not smoothly performed due to a bio-attenuation phenomenon when this technology is used to transmit data from an identification device in the body. Further, there are difficult problems of mounting an USIM chip, charging a battery, etc. when using a public wireless communication network, and hence this technology cannot be practically applied.

To solve these problems, the present invention employs a communication method such as Bluetooth or WiFi to secure low power. Specially designed BLE, WiFi HaLow, etc. for low power can be used.

The communication module of the first identification device 100 shown in FIG. 1 transmits data to the outside of the body but does not require high power because it only needs to successfully transmit data to the relay network 300 including one or more second identification devices 200, 2010, 2020, or 2030 located not so far.

The communication module of the first identification device may be set to communicate only with the second identification device 200 within the same individual, or to communicate with all the second identification devices in the communication range or the second identification devices 200 having good communication sensitivity.

The battery of the first identification device may not be easy to replace or charge, so data can be transmitted using methods to minimize battery consumption. The communication module of the first identification device can transmit data using methods such as broadcasting. The first identification device 100, for example, the communication module 120 of FIG. 2 may be set to broadcast to a distance that can be received by the second identification device (i) 200 of the same individual, by minimizing the power to transmit data. Communication modules may transmit data by broadcasting within a certain distance radius, and data may be transmitted without specifying a specific destination. Thus, a second identification device (k, n) 200 installed on other nearby individuals may receive data from the first identification device 100.

The communication module of the first identification device 100 may also be set up to enable communication with the second identification device 200 within a predetermined radius using a broadcast method. In such a case, the communication module of the first identification device may transmit bioactivity data to the second identification device (i) 2010 of the same individual or to the second identification device (n, k) 2020, 2030 located within a certain distance.

It is possible to do this setting not only reduces the amount of battery use consumed by data transmission in the first identification device but also minimizes the electromagnetic, EM, impact on individuals in the estrus, insemination, pregnancy, or delivery period of the cow.

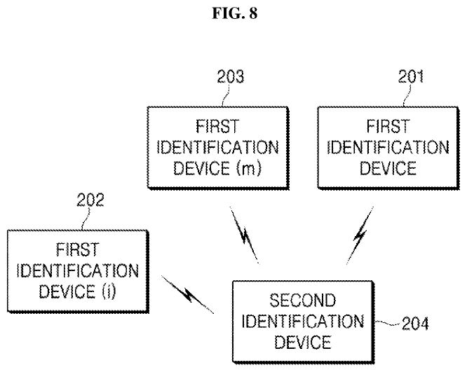

FIG. 8 illustrates an exemplary part of a configuration of a system for acquiring individual information. When the second identification device 204 is located within the range that the data may be transmitted by the first identification device, one or more first identification devices (j, l, m) 201, 202, 203 may transmit bioactivity data to the second identification device.

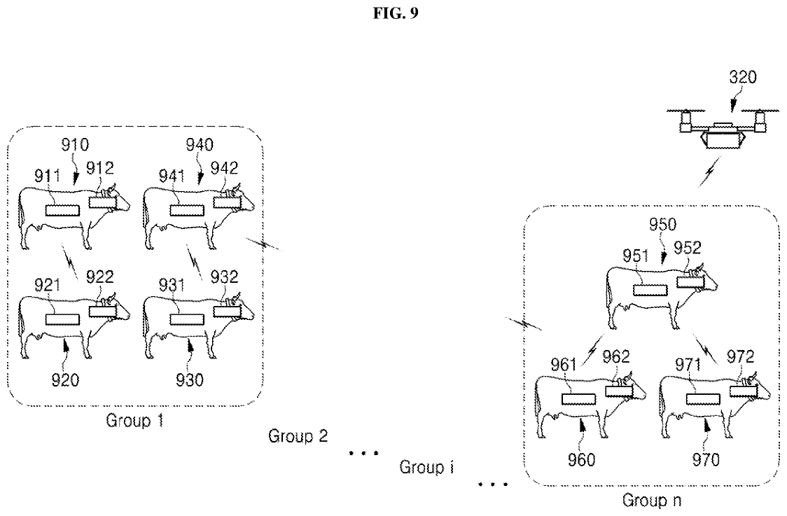

FIG. 9 illustrates an exemplary overall configuration of a system for acquiring individual information. As shown, individuals within a certain distance may be grouped in Group 1, Group 2, . . . Group i, . . . Group n by a global positioning system, GPS. For example, only the second identification device 942 among second identification devices designated in Group 1 may be powered on. The second identification device 942 may collect bioactivity information from the first identification devices installed at nearby individuals 911, 921, 931, and transmit the collected bioactivity information to a relay 320 or a base station (not in FIG. 9) along with the bioactivity information of the first identification device 941 itself.

If the area of the ranch or farm is large, with thousands of acres, and thousands of cattle must be managed, the second identification device in Group 1 may require a lot of power to transmit data to a relay 320 or a base station far away.

For example, cattle often sleep more than half a day or do not move much from their current position, so GPS information may be little changed, so the second identification devices of all individuals may not always have to manage their location.

For example, shown in FIG. 9, individuals may be grouped at a predetermined time by dividing them according to GPS information case by case.

Bioactivity information of the first identification device of each group may be collected from one or more second identification device 942, 962.

At this time, each of the first identification device may be powered on at a set time as required and the biometric information of each individual may be transmitted to the second identification device of the same individual or one or more other second identification devices of one or more other individuals close to the individual or the group that the individual is involved by Bluetooth or broadcasting.

For example, a pre-designated second identification device 922 may aggregate information of designated individuals within the same group. When the pre-designated second identification device is the second identification device 922 of cattle 920, and there is a message transmitted from the server to collect biometric information of designated individuals 910, 940 within the same group, the designated second identification device 922 may collect only these information and transmit data to a relay, a base station, or other identification devices.

The second identification device 942 may broadcast data collected within a set range nearby, or transmit the collected data to a designated relay or a designated second identification device via wireless communication such as WiFi, LTE, 5G, etc.

A second identification device may transmit data collected from a group to one or more second identification device of a neighboring group within the reach of communication, a second identification device of other predetermined groups, or predetermined one or more second identification device regardless of distance.

The second identification device of the other group (e.g., Group 2) that received the data from other groups(Group 1, Group i, or etc.)′ second identification devices may collect bioactivity information from Group 2's first identification device and transmit all data to the second identification device 962 of the neighboring group (Group n) or to the designated second identification device 972. All collected data may be transmitted to the second identification device 952 that is closest to the relay 320 or a base station (not in FIG. 9). Finally, the second identification device 952 may transmit data to a relay 320 or a base station.

If all second identification devices analyze the GPS information at short intervals to transmit the individual's location data each time and transmit the GPS information to the relay 320, etc., the power consumption may be high. In case the individual has no movement for a certain time or during a predetermined period, the second identification device may only transmit biometric information collected from the first identification devices to the relay or a base station.

For example, if an individual is not moving for a certain period or asleep, if there is no movement of rumens of individuals, or if the change in the biometric information of the individual is within a set threshold, the second identification device may not transmit data to the relay or base station if it is determined that there is no change of collected bioactivity information from the first identification devices.

### C. Authentication/Identification Module (First Individual Authentication/Identification Module, 130)

An authentication/identification module stores a first ID (i) inherent to the first identification device, and the first ID (i) is used for identification of the first identification device, identification of collected data by merging ID with the collected data, and the like.

### D. First Identification Device Control Unit 140

A first identification device control unit controls the overall operation of the first identification device, and in particular, receives data collected from the sensor module, merges the collected data with the inherent ID of the authentication/identification module, generates individual's bioactivity information data, and transmit it to the communication module.

### E. Power Control Module 150

Since the first identification device is located in the body of each individual, it is difficult to replace a battery, so there is a great need for power saving. Thus, it is necessary to put it in an inactive state when not used and to operate the device periodically or only when necessary.

Power control of the first identification device may be controlled entirely by the first identification device control unit 140. The power control module 150 may include an energy harvesting module 152 as an energy collection device, and a power activation module 154.

A power supply (battery) of the first identification device may be switched between an inactive state and an activated state according to a control command through the server—the relay network 300—the second identification device 200, 2010, 2020, or 2030, referring to FIG. 1.

The activation states can be divided into mode 1 of a high performance state and mode 2 of a normal performance state. Mode 1 may be a mode in which, when the individual is activate briskly and when the stomach or rumen of the cow is moving with digestive activity, sensors (e.g., acceleration sensor, etc.) included in the sensor module 110 within the first identification device are operated fully to measure the bioactivity information of the individual and accumulate or transmit data.

Mode 2 may be a mode that stops the measurement of sensors (e.g. the acceleration sensor) contained in the sensor module 110 or prevents the collection of data of movement of the rumen while the individual is asleep if the movement of the rumen becomes sluggish.

An inactive state may be mode 3 that may include a sleep mode in a low power state and a power cut-off mode in which power is completely cut-off.

The sleep mode may be switched to the activated state by an activation command signal, for example, by a main control unit, MCU.

While in power cut-off mode, the energy harvesting device 152 may be received an activation command message signal from the second identification device. The energy harvesting device 152 may wake up the first identification device by receiving an activation command message as a power transmission signal, converting the power transmission signal into power, entering it into the power activation module 154, and activating or enabling the power activation module 154.

For example, if the input power value is above the threshold, the MCU may set the power activation module 154 to enable mode. When the first identification device receives an activation command message from the second identification device, the sensor module 110 may be switched into the activation state.

In the power cut-off mode, an energy collection device 152 converts a power transmission signal as the activation command signal to power to activate a power activation module 154 and switch the first identification device to its activated state.

Such power control of the first identification device is overall controlled by the first identification device control unit 140, and the power control module 150 includes the energy collecting device (harvesting module 152) and the power activation module 154.

### (1) Energy Collecting Device (Harvesting Module 152)

The first identification device 100 may comprise the energy collection device. The energy collection device receives the power transmission signal transmitted from the second identification device 200, converts it into power for power activation, and transmits it to the power activation module 154.

In addition, the energy collection device may collect energy from other energy sources in the body of each individual, including, for example, electromagnetic energy, magnetic energy, kinetic energy, thermal energy, etc., and transmit it to the power activation module 154 as power for power activation.

The power transmission signal and the energy collection device can be implemented by applying conventional techniques at the time of filing.

By using the energy collection device, it is possible to efficiently operate the first identification device without consuming a limited amount of power for battery capacity, so that power of the first identification device can be effectively saved.

### (2) Power Activation Module 154

The power activation module may operate a power supply (battery) of the first identification device by using a power converted and generated by the energy harvesting device 152.

It may be configured the generated power to activates the power control module 150 that controls the battery, and the power control module 150 to activate the power supply of the first identification device 100.

The power activation module may include a switch that turns on/off the supply of battery power to the power control module through a connection between the power control module 150 and the battery.

### F. First Identification Device Memory 160

The first identification device 100 may store bioactivity information data collected in the sensor module 110, which is transmitted to the second identification device 200. If the communication environment is not smooth, the bioactivity information data may be stored in a first identification device memory prior to transmitting the data to the second identification device (i) of the same individual or the second identification devices (k) of other individuals, and then the stored data may be transmitted all at once when the communication environment is smooth.

### G. Data Structure

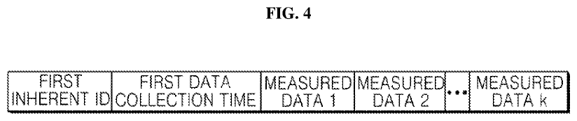

FIG. 4 is a schematic diagram showing a basic structure of data, which is identification information data, that is transmitted from a first identification device to a second identification device.

According to some embodiment, a basic structure of data transmitted from the first identification device 100 to the second identification device 200 is shown in FIG. 4. The first identification device 100 may transmit bioactivity data of an individual within a certain range through transmission, such as broadcasting.

First, a first inherent ID (i) is inputted, and then data (e.g. measured data 1, measured data 2, . . . , measured data k) measured by each sensor collected in the sensor module are included.

Measured data 1, measured data 2, . . . , measured data k may be data measured by an acceleration sensor to detect the acceleration displacement value or activity of the stomach of the cow, a gyro sensor to detect the angular velocity value of the stomach of the cow, a temperature sensor to detect the temperature value of the stomach of the cow, a methane sensor to detect the amount of methane gas produced in the stomach of the cow, and/or a PH sensor to detect the PH concentration of the stomach of the cow.

Subsequently to the first inherent ID (i), a first data collection time in which data is measured by the sensors may be included.

The first data collection time is for tracking the bioactivity of each individual over time. A time in which the sensor module 110 performs any measurement or a time in which the first identification device control unit 140 merges data may be inputted, for example.

The data of collection time may also be used, for example, to determine that identification devices do not transmit the collected data when the individual is asleep, or when the rumen is moving sluggishly.

For example, if the measured data values, measured by an acceleration sensor or gyro sensor, are expected to sustain unchanged or have been unchanged for a certain period, the second identification device may be used to determine that each measurement data for each unit of time collected is not transmitted for a predetermined period.

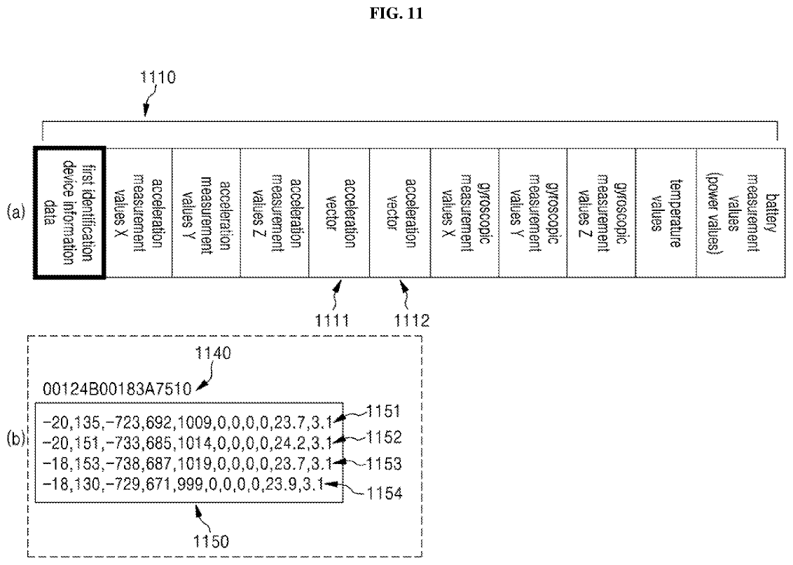

FIG. 11 illustrates an exemplary showing specific examples of identification data transmitted from a first identification device to a second identification device.

The part (a) of FIG. 11 is showing exemplary measurement data (e.g. acceleration measurement values of x, y, and z axes, the gyroscopic measurement values of x, y, and z axes) of identification information data 1110 transmitted from the first identification device to the second identification device.

The first identification device may transmit data such as the measured acceleration displacement values of x, y, and z axes to the server by using a second identification device or wireless communication.

The acceleration vector value (1111) is calculated by the measured acceleration values x, y, and z and may indicate the magnitude and direction of acceleration on each axis.

As in mathematical F=ma (F: force, m: mass, a: acceleration), the force and the acceleration value are proportional, and the acceleration vector values 1111 r_{magnitude} value can be obtained by the following Equation 1.

r_{magnitude}=\sqrt{x_{accel}²+y_{accel}²+z_{accel}²} (Equation 1)

For example, if the x_{accel} that is the force (F) in the x-axis direction, is −2 m/s², the force may be applied to the magnitude of 2 m/s² in the negative direction of the x-axis.

Acceleration vector values 1111 may be transmitted from the first identification device, but calculations may be performed on external servers to reduce power consumption after the server's receiving acceleration values x, y, z.

The acceleration interrupting value 1112 may be a critical reference that causes the first identification device to wake up in the sleep mode, etc., where the measurement is not to be performed.

In other words, if the acceleration displacement value of the first identification device exceeds a predetermined threshold, the first identification device may start the measurement with a wake-up. The number of times that the acceleration displacement value is exceeded the threshold value may be the acceleration interrupting value 1112.

For example, if the cow's rumen has not moved for a long time, the first identification device may be in sleep mode because there is no change in the measured value of the acceleration sensor. If the rumen move again, the measurement of the acceleration sensor of the first identification device may change.

If the range of changes in the measured values of the acceleration sensor exceeds a predetermined threshold, the exceeding may be called an event. The number of exceeding can be counted. The number of events may be called an acceleration interrupt value.

If such events occur more than a predetermined number of times during a set period, the sensor module of the first identification device, etc. may wake up to restart the measurement. At this time, the number of events above a predetermined threshold can be counted and transmitted as an acceleration interrupt value.

The server may analyze the received measurement data with artificial intelligence, AI, and when analyzed values are determined above or below the threshold, a process may be operated for transmitting the analysis result by an alarm to the user or for activating other predetermined processes.

The part (b) of FIG. 11 is an example of identification information data received from a first identification device when the first identification device is a bio-capsule. It can be seen that the ID 1140 of the first identification device (bio capsule) linked to the second identification device is 00124B00183A7510, and the part (b) of FIG. 11 illustrates the measured data 1150 in the bio-capsule that shows measured data 1151, 1152, 1153, 1154 in four times. Measurement data may also be measured at a narrow set time interval as needed.

### 1.2. Second Identification Device 200

According to some embodiments, a second identification device may be attached to the outside of each individual′ body to acquire location information of the individual and receive the bioactivity information from the first identification device 100.

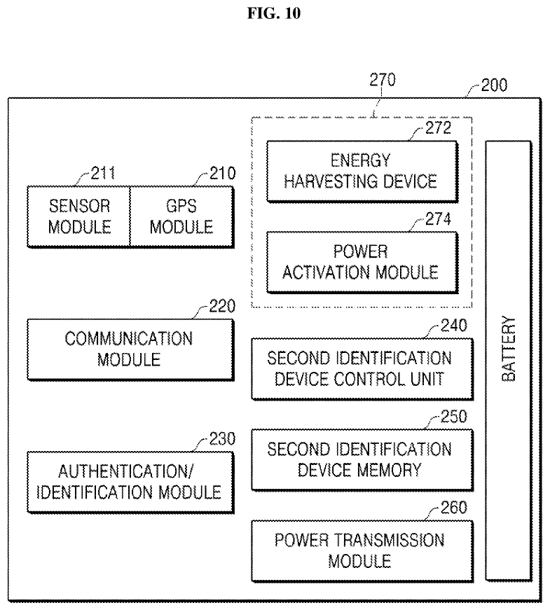

FIG. 3 or FIG. 10 are showing a schematic block diagram of a detailed configuration of a second identification device

As shown in FIG. 3, the second identification device may comprise a GPS module 210, a communication module 220, an authentication/identification module 230, a second identification device control unit 240, a second identification device memory 250, and a power transmission module 260.

The second identification device of FIG. 10 (e.g., GPS module 210) may include a sensor module 211. The second identification device may include a sensor module 211 for other measurements, besides the GPS module 210. The sensor module 211 may contain a temperature measurement sensor for measuring the body temperature or external temperature of an individual. The sensor module 211 may contain an acceleration sensor that can measure the movement and the amount of activity of an individual. The sensor module 211 may contain a sensor that can power on/off the second identification device.

As shown in FIG. 2, the second identification device comprises a sensor module 211, a communication module 220, an authentication/identification module 230, a second identification device control unit 240, and a power control module 270 including an energy harvesting module 272, and a second identification device memory 250.

A second identification device (for example, a power control module 270), including a separate energy harvesting device 272 and a power activation module 274 such a first identification device, may be configured to enable or disable either internally or externally the activation state of the second identification device.

The second identification device may include various modes, such as the sleep mode of power to shut-off completely or re wake-up a second identification device (e.g., the sensor module 211 of FIG. 10).

The mode of second identification may be configured to be externally controlled, by such as an relay, server, or base station. Depending on the results of the analysis of transmitted data by AI, the second identification device may be controlled, and the second identification device may be pre-programmed to be controlled according to the predetermined threshold value.

The GPS module 210 or sensor module 211, according to some embodiments, may contain sensors that are connected to or function with such devices, modules, or sensing functions, but not necessarily limited to them, but maybe linked to modules that include sensors to collect other information that can be provided to users or that perform various functions.

FIG. 7 is an exemplary view showing an embodiment in which first and second identification devices are attached to livestock (cows)

Contrary to the first identification device inserted in the body of each individual, the second identification device may be attached to the outside of each individual's body, as shown in FIG. 7.

A location in which the second identification device is attached is not limited, and as an example, it may be attached to each individual's ear as an ear tag or as a necklace.

The system according to some embodiments comprises a plurality of second identification devices 200 (second identification device (i), i=1, 2, 3 . . . , k=the total number of livestock).

### A. GPS Module 210

The second identification device includes a GPS module for acquiring location information of each individual from the outside of the individual's body.

The GPS module receives current location information from a satellite and transmits the data to a second identification device control unit 240 as will be described below.

### B. Communication Module (Second Individual Communication Module, 220)

A communication module of the second identification device communicates with the first identification device in the body and a relay network 300 composed of a first mobile relay 320 such as a drone and a second relay 340 such as a balloon/ground device.

The communication module of the second identification device is a communication module for transmitting bioactivity information data acquired via a communication with the first identification device 100 and location data acquired in the GPS module 210 to the outside (relay network 300).

The relay network 300 may include the second identification devices 200, and the communication module of the second identification device transmits data transmitted from the second identification device control unit 240 to the relay network 300 composed of other second identification devices, the first relay 320 and the second relay 340.

### (1) Data Communication Path

The first identification device 100 has at least two communication paths. A first communication path is a communication path for data transmission/reception between the first identification device 100 and the second identification device 200, and a second communication path is a communication path for a communication with the second identification device 200 and the relay network 300.

### (2) Data Communication Mode

The communication module of the second identification device 200 supports at least two communication modes.

A first communication mode uses a low-power Bluetooth or WiFi communication mode to communicate with the first identification device 100, and a second communication mode is a wireless communication mode to transmit data to the relay network 300. The second communication mode may use a public communication network, or may adopt a separately designed long-distance wireless communication mode.

The communication module of the second identification device can also use the first communication mode when transmitting data to the relay network 300. In this case, after receiving data, the first and second relays of the relay network 300 transmit the data to a base station using the second communication mode.

The communication module 220 of the second identification device (i) may receive not only bioactivity information data of the first identification device (i) of the same individual, but also bioactivity information data of the first identification devices (k) of other individuals.

(3) Method for Selecting Data from the First Identification Device

To allow for the second identification device (i) to selectively receive data from the first identification device, two methods may be used.

In a first method, the second identification device receives bioactivity information data only from the first identification device (i) of the same individual and excludes data from the first identification devices (k) of other individuals. The first method may be performed by setting a communication path via authentication on whether a first ID of data received from the first identification device is a matched ID in an authentication/identification module 230. The communication path with the communication module 120 of the first identification device may be set via a selective Bluetooth/WiFi connection rather than an open mode.

Also, in the first method, when setting a mutual communication path by the first identification device 100 and the second identification device 200, the communication path may be set between mutually authenticated identification devices through a Bluetooth/WiFi device connection method.

In a second method, the second identification device receives a signal having good communication sensitivity regardless of whether or not the same individual. For example, when the second identification device (i) is an ear tag attached to the ear of each individual, better communication sensitivity may be obtained by entering a distance adjacent to the first identification device (i+1) in the body of another individual. In this case, the second identification device (i) transmits/receives data by setting a communication path with the first identification device (i+1) having good communication sensitivity.

### C. Authentication/Identification Module (Second Individual Authentication/Identification Module, 230)

An authentication/identification module of the second identification device stores a second ID (i) inherent to the second identification device, and the second inherent ID (i) is used for identification of the second identification device, identification of collected data by merging ID with the collected data, and the like.

When the first method of ‘Method for selecting data from the first identification device 100’ is applied, the authentication/identification module of the second identification device may extract the first ID (i) of the first identification device contained in a first data (bioactivity information) transmitted from the first identification device 100 and identify whether the data is data from the first identification device pre-matched with the second identification device (i).

In this case, the second identification device 200 may set a near-field communication path only with the first identification device with which is pre-matched in a communication connecting step.

### D. Second Identification Device Control Unit 240

A second identification device control unit controls the overall operation of the second identification device 200, and in particular, receives data collected from the first identification device 100 and the GPS module 210, merges the collected data with the inherent ID of the authentication/identification module 230, generates individual's identification information data, and transmit it to the communication module 220.

### E. Data Structure

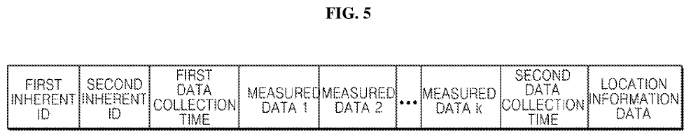

A basic structure of data transmitted from the second identification device 200 to the relay network 300 is shown in FIG. 5.

First, a second inherent ID (i) is inputted, and then data measured by each sensor collected in the sensor module or location information data acquired in the GPS module are included. A first data collection time in which data is measured by the sensors and a second data collection time contained in the location information may be included.

The first and second data collection times are for tracking bioactivity of each individual over time. A time in which the sensor module 110, 211 performs any measurement or a time in which the first identification device control unit 140 or the second identification device control unit 240 merges data may be inputted, for example. Alternatively, a time data contained in the GPS location information data may be inputted.

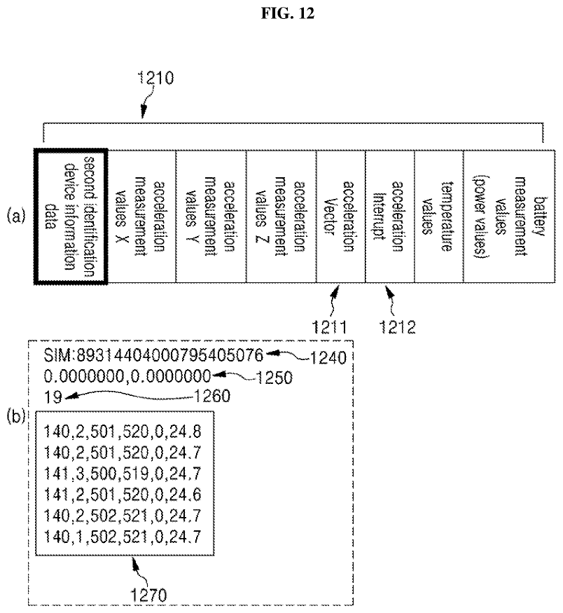

The part (a) of FIG. 12 illustrates an exemplary specific example of identification information data measured by a second identification device.

The second identification device may transmit measurement data (e.g. acceleration measurement values of x, y, and z axes, the gyroscopic measurement values of x, y, and z axes) of identification information data 1210 along with bioactivity data received from the first identification device (e.g., identification information data of FIG. 11) to a relay, base stations, or a second identification device of other individuals.

The second identification device may transmit data such as the measured acceleration displacement values of x, y, and z axes to the relay or the server by broadcasting or wireless communication.

The acceleration vector value (1211) is calculated by the measured acceleration values x, y, and z and may indicate the magnitude and direction of acceleration on each axis.

As in mathematical F=ma (F: force, m: mass, a: acceleration), the force and the acceleration value are proportional, and the acceleration vector values 1111 r_{magnitude} value can be obtained by the following Equation 2.

r_{magnitude}=\sqrt{x_{accel}²+y_{accel}²+z_{accel}²} (Equation 2)

For example, if the x_{accel} that is the force (F) in the x-axis direction, is −2 m/s², the force may be applied to the magnitude of 2 m/s² in the negative direction of the x-axis.

Acceleration vector values 1211 may be transmitted from the second identification device, but calculations may be performed on external servers to reduce power consumption after the server's receiving acceleration values x, y, z.

The acceleration interrupting value 1212 may be a critical reference that causes the second identification device to wake up in the sleep mode, etc., where the measurement is not to be performed.

In other words, if the acceleration displacement value of the second identification device exceeds a predetermined threshold, the second identification device may start the measurement with waking-up. The number of times that the acceleration displacement value is exceeded the threshold value may be the acceleration interrupting value 1212.

For example, if the cow has not moved for a long time, the second identification device may be in sleep mode because there is no change in the measured value of the acceleration sensor. If the cow moves again, the measurement of the acceleration sensor of the second identification device may change.

If the range of changes in the measured values of the acceleration sensor exceeds a predetermined threshold, the exceeding may be called an event. The number of exceeding can be counted. The number of events may be called an acceleration interrupt value.

If such events occur more than a predetermined number of times during a set period, the sensor module of the second identification device, etc. may wake up to restart the measurement. At this time, the number of events above a predetermined threshold can be counted and transmitted as an acceleration interrupt value.

The server may analyze the received measurement data with AI, and when analyzed values are determined above or below the threshold, a process may be operated for transmitting the analysis result by an alarm to the user or for activating other predetermined processes. The part (b) of FIG. 12 is an example of identification information data transmitted from a second identification device when the second identification device is an ear tag. It can be seen that the ear tag's ID 1240 of the second identification device (e.g., ear tag or necklace) linked to the second identification device is 89314404000795405076, and the part (b) of FIG. 12 illustrates the measured data 1270 in the ear tag that shows measured data in six times. Measurement data may also be measured at a narrow set time interval as needed.

### F. Second Identification Device Memory 250

The second identification device includes a second identification device memory 250 for storing individual identification information data generated by the second identification device control unit 240. Accordingly, if desired, the individual identification information data may be obtained from the second identification device by separating the second identification device (i) from the corresponding individual (i).

Also, it is possible to store individual data transmitted from other second identification devices.

In addition, it is possible to store various additional information for operation of the second identification device as set by a user (farm owner/keeper).

### G. Power Transmission Module 260

The second identification device may include a power transmission module to activate the first identification device 100.

The power transmission module receives an activation command through the base station 400—the relay network 300 and transmits this signal to the first identification device 100 via the communication module 220 of the second identification device.

The activation command signal may be a simple control signal for waking up the first identification device in an inactive state. When the first identification device is in a completely OFF state, the first identification device is activated by transmitting a power transmission signal as the activation command signal or along with the activation command signal.

Accordingly, as previously described, the energy collection device 152 of the first identification device, which receives the power transmission signal as the activation command signal transmitted from the power transmission module, converts the power transmission signal into power for power activation to drive a power supply (battery).

### H. Data Generation Cycle and Synchronization

According to some embodiments, the generation period of a first data (bioactivity information data collected in the first identification device) and a second data (location information data collected in the second identification device) may be set to a predetermined time period and synchronized.

For the convenience of data collection/management in a server, a predetermined time period is set to 30 secs, for example, and a synchronization clock signal is transmitted from the second identification device control unit to the first identification device to synchronize bioactivity information collection time with GPS information collection time.

The synchronization clock signal may be generated in the second identification device control unit 240 as well as the first identification device control unit 140 and the server, and may be transmitted to the first and second identification devices 100 and 200.

### 1.3. Relay Network 300

According to some embodiments, a relay network receives data from the first and second identification devices 100 and 200 and transmits the data to a base station.

The relay network may include all or selectively a first relay 320 and a second relay 340, and may further include second identification devices.

### A. First Relay 320

A first relay may be configured as a mobile communication device, typically a mobile flight vehicle (drone).

The first relay receives individual information including individual's bioactivity and location information from the second identification device 200 and transmits it to a base station 400. The first relay transmits the individual information to the base station 400 using a conventional wireless communication method.

Besides a drone, the first relay may be mounted on a vehicle that follow livestock in grazing land as well as an unmanned small vehicle, a robot, etc. to move along livestock in grazing land.

The first relay may have a first relay memory (not shown) to select whether transmitting individual information received from the second identification device 200 to the base station or storing it in the memory. If the individual information is stored in the memory, it may be managed by collecting data from the memory at the base station when the first relay is recovered.

### B. Second Relay 340

A second relay may be configured as a ground communication device. It may be fixedly installed at a predetermined location in grazing land. When the second identification device 200 is within a communication range, the second relay collects individual information from the second identification device 200 and transmits it to the base station 400.

The second relay and the base station 400 may be connected through a wired/wireless network.

As previously described for the first relay 320, the second relay may also include a second relay memory (not shown) to store received data.

### C. Network Comprised of One or More Second Identification Devices

The second identification devices may function as relays. In this case, the second identification devices may perform a function of receiving data from other second identification devices and transmitting the data to the first or second relay 320 and 340.

The first and second relays 320 and 340 may not be able to communicate with some or all of the second identification devices due to a change in communication quality due to the influence of distance/attenuation.

To solve this problem, the second identification devices receive and store individual information from other second identification devices, and then transmit such information to the first and second relays 320 and 340 within a communication range.

Accordingly, the second identification devices may form various network structures such as a ring shape and a star shape, including the first and second relays 320 and 340. Also, the second identification devices may have an ad-hoc network type that is actively connected to a network according to communication quality. In this case, data which is transmitted to the first and second relay by the second identification device is constructed as follows, for example.

### (1) Transmission Data Construction

The second identification device (i) may receive data from other second identification devices (k) and hold it along with its own bioactivity information data.

At this time, the individual data (i) of the second identification device (i) may be constructed as “first ID (i)-individual bioactivity information (i)-second ID (i)-individual location information (i)-time stamp”, and the individual data (k) of other second identification device (k) may be constructed as “first ID (k)-individual bioactivity information (k)-second ID (k)-individual location information (k)-time stamp”.

The second identification device (i) receives the individual data (k) of the second identification device (k) and stores it in the memory. Thereafter, when the second identification device (i) is in a position which is able to communicate with the first and second relays 320 and 340, the second identification device (i) may transmit its own individual data (i) along with the individual data (k) from other second identification device (k) stored in the memory.

The data thus transmitted may be transmitted back to other second identification device (n). Through this process, the second identification device (i) of an individual at the outermost position of livestock herd in grazing land may receive and hold individual data from the second identification device (k) on the opposite side or the center of livestock herd as far as the data capacity allows.

For example, the first relay (drone) may collect data of individuals in the center or the opposite side even in the outermost position of the herd and transmit the collected data to a base station or store it in the memory without disturbing the herd in the center.

### (2) Second Identification Device Memory

According to some embodiments, the second identification device has a temporary memory for the construction of transmission data and storage/transmission thereof. As far as the memory capacity allows, the second identification device receives and stores individual data transmitted from other second identification devices within a communication range, and then transmits the data to the first and second relays.

### D. Effects of the Relay Network According to Some Embodiments

As described above, according to some embodiments includes, the second identification device is included in the relay network, as well as the second identification device receives and stores individual data from other second identification devices, and then transmits the data to the first and second relays. Thus, the present invention can solve the problem that a conventional relay system does not receive data stably.

Even if a mobile relay is introduced to solve the problem of a fixed relay, there is a high possibility that the mobile relay does not always communicate with the second identification device. To solve this problem, according to some embodiments, after the second identification device receives and stores data from other second identification device, when the first and second relays are within a communication range, the second identification device transmits its own data along with data from the other second identification devices. Thus, as described above, the second identification device in the outermost position may receive and hold individual data from the second identification device in the center, or may transmit the data to the first and second relay.

### 1.4. Base Station 400

According to some embodiments, a base station may be installed in any site such as a ranch/farm, or a data management center located at a remote place.

The base station is connected to the first and second relays 320 and 340 via a wired/wireless communication to collect individual information data. Also, the individual information data may be collected from a memory (not shown) of the first relay 320 when the first relay 320 is recovered. In addition, data may be collected and verified from a memory (not shown) of the second relay 340.

A data processing server is connected to the base station, and such a data processing server and data management are according to conventional data processing methods.

### 1.5. Client (e.g., a Client 500 of FIG. 1)

According to some embodiments, the system may include a client 500 as a user's terminal.

A client 500 may include the base station 400, a data processing center, or server (not shown) for operation, and may be further provided as a mobile terminal so that a farmer/keeper etc. can read data such as individual's location/bioactivity information.

Now, a method for acquiring individual information according to some embodiments will be described.

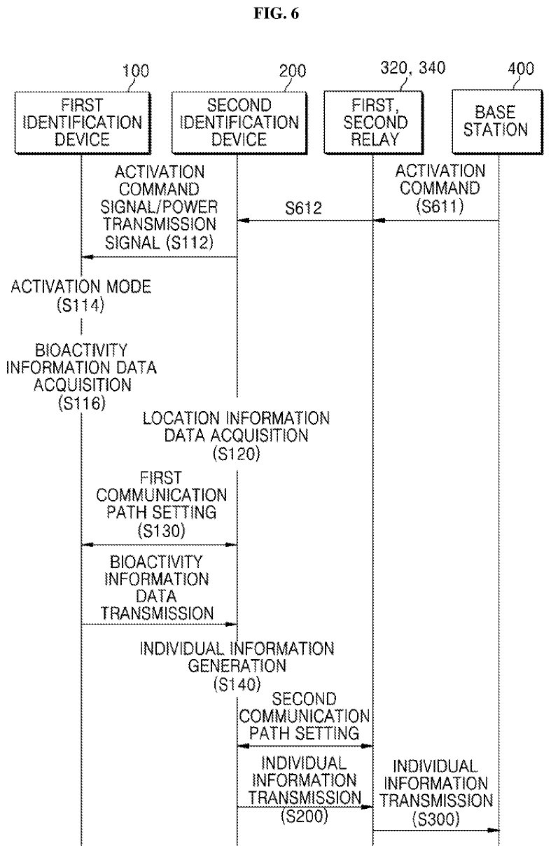

FIG. 6 is a flowchart illustrating a method for acquiring individual information.

The method for acquiring individual information comprises: an individual data acquisition step (S100) in which data is acquired from each individual; a relay network transmission step (S200) in which the acquired individual data is transmitted to a relay network; and a base station transmission step (S300) in which the individual data is transmitted from the relay network to a base station.

Hereinafter, each of the steps will be described.

### 2.1. Individual Data Acquisition Step (S100)

The term ‘a second identification data acquisition step (S120)’ is interchangeably used with ‘location information data acquisition (S120)’ of FIG. 6.

The term ‘a communication path is set between the first identification device and the second identification device (S130)’ is interchangeably used with ‘first communication path setting (S130)’ of FIG. 6.

Even figure No. of S110 is not shown in FIG. 6 but the step of ‘a first identification data acquisition step (S110)’ may be configured to include at least one of steps of S611, S612, S112, S114, or S116.

The term ‘an individual data generation step (S140)’ is interchangeably used with ‘individual information generation (S140)’ of FIG. 6

The individual data acquisition step comprises: A) a first identification data acquisition step (S110) in which a first identification data is acquired through a sensor module in a first identification device; B) a second identification data acquisition step (S120) in which a second identification data is acquired through a GPS module in a second identification device; C) a first communication path setting step in which a communication path is set between the first identification device and the second identification device (S130); and D) an individual data generation step (S140) in which the first identification data is received from the second identification device and is integrated with the second identification data to generate individual data.

### A. First Identification Data Acquisition Step (S110)

When the first identification device is in an inactive state, the first identification data acquisition step may be started from a first identification device activation step which activates the first identification device.

### (1) Activation Command Transmission Step (S112)

If an activation command S611 is transmitted from a server, it is transmitted to the second identification device 200 through the base station 400—the relay 300. Accordingly, the communication module 220 of the second identification device (i, for example 2010 of FIG. 1) transmits the activation command signal to the first identification device (i).

The activation command signal may be a simple control signal for waking up the first identification device in an inactive state. When the first identification device is in a completely OFF state, the first identification device is activated by transmitting a power transmission signal as the activation command signal or along with the activation command signal. (Power transmission module, 260)

The first identification device 100 may switch an activation/inactivation state of the device according to a predetermined period or time schedule without an activation command signal from the second identification device 200.

### (2) First Identification Device Activation Step (S114)

In this step, the first identification device 100 is activated by receiving an activation command signal. When the first identification device receives a control signal as the activation command signal, a sleep mode is switched to an activation mode. When the first identification device receives a power transmission signal as the activation command signal, a power cut-off mode is switched to an activation mode.

### (3) Bioactivity Information Data Acquisition Step (S116)

In this step, bioactivity information data is acquired from each individual through the sensor module of the first identification device in an activated state. The bioactivity information data is stored as the first identification data along with an inherent ID of the first identification device, and is transmitted to the second identification device.

### B. Second Identification Data Acquisition Step (S120)

In this step, location information is acquired from each individual through the GPS module 210 in the second identification device 200. The acquired location information is stored as the second identification data along with an inherent ID of the second identification device 200.

### C. First Communication Path Setting Step (S130)

In this step, a communication path is set between the first identification device 100 and the second identification device 200.

As previously described, the first communication path between the first identification device 100 and the second identification device 200 is preferably set using low-power Bluetooth (BLE)/Wi-Fi communication technologies, but is not limited thereto.

The first communication path may be set only between the predetermined first and second identification devices 100 and 200. Alternatively, in an open situation, a communication path may be set between the first and second identification devices 100 and 200 with good signal sensitivity.

Referring to FIG. 6 and FIG. 8, the first communication path setting S130 may be configured to set between the second identification device 204 and one or more of the first identification devices 201, 202, 203. The data including bioactivity data may be transmitted from one or more first identification devices 201, 202, 203 to the second identification device 204.

Each first identification device 201, 202, 203 may be located in a different individual. One of the first identification devices 201, 202, 203 may be installed in the same individual with the second identification device 204.

### D. Individual Data Generation Step (S140)

After the first communication path is set between the first identification device 100 and the second identification device 200 in the first communication path setting step (S130), the second identification device 200 receives the first identification data from the first identification device 100.

The received data is merged in the second identification device 200, or alternatively, individual information (data) is generated from each data along with inherent IDs of the first and second identification device 100 and 200 and a time stamp. Then, the resulting data is stored.

As shown in FIG. 8, the second identification device 204 may also receive the first identification data from the first identification device which do not belong to the same individual.

### 2.2. Relay Network Transmission Step (S200)/Base Station Transmission Step (S300)

The individual data acquired in the individual data acquisition step (S100) is transmitted to a base station via a relay network. The steps of transmitting from the first identification device 100 to the base station 400 are as follows.

### A. Second Communication Path Setting Step (Relay Network Construction)

The second identification device (i) constitutes a relay network 300 including other second identification devices (k) and/or a first relay 320 and/or a second relay 340.

### B. Individual Information Transmission Step

The second identification device 200 transmits the generated individual information to the relay network 300. As previously described, the relay network 300 may include second identification devices (i, k, n, . . . ), in this case, the second identification device (i) receives and stores individual information transmitted from other second identification devices (k, n, . . . ), and then transmits this information to the first and/or second relays 320 and 340 (S200).

The individual information received from the second identification device is transmitted to the base station 400 through the first and/or second relays 320 and 340, and hence the transmission of individual information is completed (S300).

### C. First Identification Device Inactivation Step

After transmitting the identification data, the first identification device 100 may be inactivated for battery management. Such inactivation may have a sleep mode and/or a power cut-off state.

The first identification device may be switched between activation and inactivation according to a predetermined timer setting, and according to an activation/inactivation command via the server—the relay 300—the second identification device 200.

Although the technical concept of the present invention has been described in detail with reference to the embodiments set forth above, it should be noted that these embodiments are for the purpose of explanation and not for the limitation thereof. Furthermore, it can be appreciated by one with ordinary skill in the art that various variations are possible within the spirit and scope of the present invention.

### NUMERICAL NUMBER DESCRIPTION

100, 911, 921, 931, 941, 951, 961, 971: first identification device
110: sensor module
120: communication module
130: authentication/identification module
140: first identification device control unit
150: power control module
152: energy harvesting device
154: power activation module
160: first identification device memory
200, 912, 922, 932, 942, 952, 962, 972: second identification device
210: GPS module
220: communication module
230: authentication/identification module
240: second identification device control unit
250: second identification device memory
260: power transmission module
270: power control module
300: relay network
320: first relay
340: second relay
400: base station
500: one or more clients

What is claimed is:

1. A system for acquiring individual information including bioactivity and location information from two or more individuals, the system comprising:
a first identification device inserted in the body of each individual to acquire bioactivity information of the individual;
a second identification device attached to the outside of each individual's body to acquire location information of the individual and to receive the bioactivity information acquired by the first identification device for generating individual information including the bioactivity and location information; and
a relay network to receive individual information including the bioactivity and location information from the second identification device.

2. The system for acquiring individual information according to claim 1, wherein the second identification device further receives bioactivity and/or location information from other second identification devices of other individuals.

3. The system for acquiring individual information according to claim 2, wherein the relay network includes the second identification device of each individual.

4. The system for acquiring individual information according to claim 1, wherein the first identification device has an activation mode and an inactivation mode, and switches between the activation mode and the inactivation mode according to a control signal transmitted from the second identification device.

5. The system for acquiring individual information according to claim 4, wherein the inactivation mode of the first identification device has a power cut-off mode in which power is completely cut-off, and the power cut-off mode is switched to the activation mode due to a power transmission signal transmitted from the second identification device.

6. The system for acquiring individual information according to claim 3, wherein the second identification device transmits its own individual information along with individual information received from other second identification devices to the relay network.

7. The system for acquiring individual information according to claim 1, wherein the first identification device has an activation mode and an inactivation mode, and switches between the activation mode and the inactivation mode according to a predetermined time-period.

8. A method for acquiring individual information including bioactivity and location information from two or more individuals, the method comprising:
acquiring bioactivity information of each individual by a first identification device inserted in the body of the individual (bioactivity information-acquiring step);
acquiring location information of the individual and receiving the bioactivity information from the first identification device by a second identification device attached to the outside of each individual's body for generating individual information including the bioactivity and location information (individual information-generating step); and
transmitting the generated individual information to a relay network (individual information-transmitting step).

9. The method for acquiring individual information according to claim 8, wherein the individual information-transmitting step further comprises receiving individual information from other second identification devices of other individuals and transmitting it.

10. The method for acquiring individual information according to claim 8, further comprising activating the first identification device prior to the bioactivity information-acquiring step (first identification device-activating step).

11. The method for acquiring individual information according to claim 10, wherein the first identification device-activating step receives an activation command including a power transmission signal, and the first identification device converts the power transmission signal into power and supplies power required to activate the first identification device.

12. The system for acquiring individual information according to claim 1, wherein the second identification device further receive bioactivity information from one or more other first identification devices of other individuals.

13. The method for acquiring individual information according to claim 8, wherein the individual information-transmitting step further comprises receiving individual information from one or more other second identification devices of other individuals and transmitting it.

14. The method for acquiring individual information according to claim 8, wherein the bioactivity information-acquiring step further comprises receiving bioactivity information from one or more other first identification devices of other individuals and transmitting it.
